(19) United States
(12) Patent Application Publication
(10) Pub. No.: US 2005/0202080 A1
(43) Pub. Date: Sep. 15, 2005

(54) In process conversion method for preparing tannate tablet, capsule or other solid dosage forms

(76) Inventors: Emily C. Ware, Athens, GA (US); Jeffrey S. Kiel, Gainesville, GA (US); H. Greg Thomas, Villa Rica, GA (US); Brady Neal Ware, Athens, GA (US); George T. Harned III, Oakwood, GA (US)

Correspondence Address: KING & SCHICKLI, PLLC, 247 NORTH BROADWAY, LEXINGTON, KY, 40507, US

(21) Appl. No.: 11/078,854

(22) Filed: Mar. 11, 2005

Related U.S. Application Data
(60) Provisional application No. 60/552,519, filed on Mar. 12, 2004.

(51) Int. Cl. A61K 31/7024; A61K 9/20; A61K 9/48

(52) U.S. Cl. 424/451; 514/23; 424/464

(57) ABSTRACT

The present invention relates generally to the field of tannate chemistry and more specifically to methods for processing tannate tablets, capsules, or other solid dosage forms. The present invention provides a novel manufacturing process for the conversion of one or more active pharmaceutical ingredients (“API”) into their tannate salt complexes while incorporating the complexes into a therapeutic solid-dosage form which also may include non-tannate API's. The first step of this process is to create a tannic acid powder blend by combining the salt or free base form of one or more APIs with tannic acid. After the dry blend is thoroughly mixed, a pharmaceutically acceptable liquid is added, for example by spraying, onto the dry powder blend facilitating the tannate salt conversion process. The conversion product is then added to additional dry powders thereby reducing the overall liquid content to a level that is more typical of wet granulation processes.

This application claims the benefit of U.S. Provisional Patent Application Ser. No. 60/552,519 filed on Mar. 12, 2004.

### BACKGROUND OF THE INVENTION

1. Field of the Invention.

The present invention relates generally to the field of tannate chemistry and more specifically to methods for processing tannate tablets, capsules, or other solid dosage forms.

2. Description of the Prior Art.

The use of tannate salts in pharmaceutical preparations is well-known. Tannate salts afford a prolonged release of the active pharmaceutical ingredient and such solid dosage forms are needed to improve patient compliance with dosage requirements. U.S. Pat. No. 6,287,597 describes tannate suspensions containing pyrilamine tannate and phenylephrine tannate. The suspension is prepared in a conventional manner such that one teaspoon contains 30 mg pyrilamine tannate and 5 mg phenylephrine tannate agent, natural and artificial flavors, glycerin, kaolin, magnesium aluminum silicate, methyl paraben, pectin, purified water, saccharin, sodium hydroxide, and sucrose or sorbitol. The January 1990 issue of Annals of Allergy, Volume 64, describes combinations of chlorpheniramine tannate, pyrilamine tannate and phenylephrine tannate. An article in Clinical Medicine, dated September 1965, pages 1475-1478, describes tablets of pyrilamine tannate, chlorpheniramine tannate, and amphetamine tannate. Phenylephrine tannate compositions are disclosed in U.S. Pat. No. 5,599,846 and phenylephrine tannate and chlorpheniramine tannate compositions are disclosed in U.S. Pat. No. 6,037,358. None of these references suggest or describe the production of a solid dosage form by means of an in process conversion of the active ingredient to the tannate salt complex using the method described herein to provide a dosage form which affords a sustained release of the active ingredient over prolonged intervals of time. In addition, none of these references describe a solution to the inherent difficulties encountered in preparing tannate pharmaceutical products. Because of the size of the tannic acid molecule, the percentage of active free-base within the tannate salt is significantly lower than that in other salt forms such as the hydrochloride or maleate. The variable purity of the commercially available tannate salts leads to variation in the stoichiometry of the active free-base to tannic acid in the tannate salts from batch to batch. The low percentage of active free-base in the tannate salt exacerbates the problems associated with variable stoichiometry in the commercially available tannate salts. This problem was noted in U.S. Pat. Nos. 5,599,846 and 5,663,415. This causes significant processing problems during manufacture and increases the likelihood that commercially available pharmaceutical products contain variable and in some instances, sub-therapeutic levels of the active drug substances creating dosing problems. Therefore, it would be desirable if solid dosage form pharmaceutical compositions containing tannate salts of active ingredients could be prepared with reduced variability in active drug content and increased certainty that the active drug is delivered within the therapeutic range. The present invention provides an economical solution to the preceding difficulties. The present invention is particularly well suited for those applications where the active pharmaceutical ingredient can not easily be converted to a tannate salt without being solubilized.

### SUMMARY OF THE INVENTION

The present invention provides a novel manufacturing process for the conversion of one or more active pharmaceutical ingredients (“API”) into their tannate salt complexes while incorporating the complexes into a therapeutic solid-dosage form which also may include non-tannate API's. By starting with a commonly available salt or free base of the active pharmaceutical ingredient, which is subsequently converted and incorporated in-process as a tannate salt complex, the invention provides an efficient and reproducible method to manufacture tablet, capsule, or other solid dosage form products containing tannate salt complexes as active ingredients with decreased variability in dose.

The process provides for the addition of a pharmaceutically acceptable liquid to a powder mixture of one or more active pharmaceutical ingredients, one or more dispersing agents, and tannic acid which generates tannate salt complexes. The presence of the dispersing agent prevents the clumping and aggregation of the tannate salt complex formed. Without further treatment the tannate salt complex of one or more API's may then be combined with pharmaceutically acceptable excipients such as diluents, binders, lubricants, glidants, coloring, sweetening and flavoring agents and processed into suitable solid-dosage forms. The tannate salt complexes of the active ingredient may afford absorption of the active over prolonged intervals of time. In addition, tannate salts have been found to have better organoleptic properties in comparison to other salts or freebase forms.

Naturally occurring tannic acid comprises a mixture of compounds. They are considered to be secondary metabolites, with a molecular weight of 500-5000 Da, that have no specific metabolic function. As with many natural polymers, a rigorous chemical definition of tannins is difficult.

Hydrolyzable tannins are molecules with a polyol (generally D-glucose) as a central core, with the hydroxyl groups of the carbohydrate partially or totally esterified with phenolic groups. They derive their name from their propensity to be hydrolyzed by mild acids or mild bases to yield carbohydrates and phenolic acids. Synthetic tannic acid may comprise a purified form of any of the components of naturally occurring tannic acid.

The present invention may utilize tannic acid of either a natural or synthetic source. The term “tannic acid” herein refers to either natural or synthetic tannic acid as described above.

By forming the tannate salt complex of one or more active pharmaceutical ingredients in-situ, removing the necessity of an additional isolation step, the invention provides an efficient and reproducible method to manufacture solid-dosage forms that solves the problems referenced above in the prior art. The invention enables the production of finished pharmaceutical products which also may afford a prolonged release of active pharmaceutical ingredients, thereby reducing the frequency of drug administration and improving patient compliance.

### DETAILED DESCRIPTION OF THE INVENTION

The present invention provides a novel manufacturing process for the conversion of one or more active pharmaceutical ingredients (“API”) into their tannate salt complexes while incorporating the complexes into a therapeutic solid-dosage form which also may include non-tannate API's. The first step of this process is to create a tannic acid powder blend by combining the salt or free base form of one or more APIs with tannic acid. One or more anti-clumping agents and other pharmaceutically acceptable ingredients such as diluent may also be added to the mixture. The presence of the anti-clumping agent prevents the aggregation of the tannate salt complex formed and promotes uniformity in the powder blend. After the dry blend is thoroughly mixed, a pharmaceutically acceptable liquid is added, for example by spraying, onto the dry powder blend facilitating the tannate salt conversion process. The pharmaceutically acceptable liquid may include a binder agent. The mixture is blended with the pharmaceutically acceptable liquid and additional liquid is added until a dough-like consistency is obtained. This process solubilizes the API and tannic acid enough to facilitate conversion of the API to the tannate salt. This mixture is herein referred to as the “conversion product”. However, the dough-like consistency of the conversion product is not typical of wet granulation processes, and creates significant problems in production. The present invention overcomes this problem by directly adding the conversion product to additional dry powders thereby reducing the overall liquid content to a level that is more typical of wet granulation processes. The additional dry powders may include any one or more pharmaceutically acceptable ingredients suitable for use during wet granulation processes. The amount of liquid used as well as the amount of dry powder added is selected so that 1) the API is sufficiently solubilized in order to convert to the tannate salt form and 2) the final blend after the addition of the dry powders has a moisture content more typical of wet granulation processes. Blending of the dry powders and the conversion product is most preferably accomplished using a paddle or ribbon type blender. The resultant mixture may then be processed in a manner typical of wet granulations.

Naturally occurring tannic acid comprises a mixture of compounds. They are considered to be secondary metabolites, with a molecular weight of 500-5000 Da, that have no specific metabolic function. They are complex phenol-rich polymers found in many foods. As with many natural polymers, a rigorous chemical definition of tannins is difficult. In general two classes are distinguished—the hydrolyzable and the condensed tannins. Hydrolyzable tannins or tannic acids are referenced in the various pharmacopeias and are composed of gallic acid or its condensation product ellagic acid esterified to the hydroxyl groups of glucose.

Hydrolyzable tannins are molecules with a polyol (generally D-glucose) as a central core, with the hydroxyl groups of the carbohydrate partially or totally esterified with phenolic groups. They derive their name from their propensity to be hydrolyzed by mild acids or mild bases to yield carbohydrates and phenolic acids. Synthetic tannic acid may comprise a purified form of any of the components of naturally occurring tannic acid.

The present invention may utilize tannic acid of either a natural or synthetic source. The term “tannic acid” herein refers to either natural or synthetic tannic acid as described above.

The conversion process requires the presence of basic functional groups such as amines and sulfoxides in the molecular structure of the API. The formation of the tannate salt is by reaction of amine groups (in the 1°, 2°, 3°, 4°, or amphoteric functional states) or of the other basic function groups with tannic acid. The amount and ratio of dispersing agent and tannic acid required for the completion of the reaction is determined by the molecular configuration and concentration of the API.

The API tannate salt complex or complexes obtained from the above conversion process are mixed with one or more of the following: diluent, binder, lubricant, sweetening, hardness increasing, coloring, flavoring, preservative, suspending, stabilizing, and flow enhancing agents as necessary. The granulate produced by this method is processed into tablet, capsule or other solid-dosage forms as necessary using techniques well known in the art.

By starting with a known amount of commonly available salt or the free base form of the API which is subsequently converted and incorporated as a tannate salt complex into a solid dosage form without additional isolation, the invention provides an efficient and reproducible method to manufacture products containing tannate salt complexes as active ingredients. Since the tannate salt complex of the API is generated and incorporated into the dosage form during the manufacturing process, the need to isolate the tannate salt is eliminated and the stoichiometry of the tannate salt is uniform from batch to batch resulting in decreased dosage variability.

The following is a non-exclusive list of active pharmaceutical ingredients that may be used in this process:
1. carbinoxamine
2. chlorpheniramine
3. dexchlorpheniramine
4. phenazopyridine
5. pyrilamine
6. pheniramine
7. diphenhydramine
8. bromodiphenhydramine
9. tripelennamine
10. brompheniramine
11. loratadine
12. desloratadine
13.fexofenadine
14. carbetapentane
15. dextromethorphan
16. phenylephrine
17. pseudoephedrine
18. ephedrine
19. oxycodone
20. morphine
21. physostigmine
22. cimetidine
23.amantidine
24.fluvoxamine
25. sertraline
26. chlorpromazine
27. imipramine
28.amitryptyline
29. prochlorperazine
30. cetirizine
31. hydroxyzine
32. promethazine
33. acrivastine
34.triprolidine
35. meclizine
36.dimenhydrinate
37. doxylamine
38. diphenylpyrilamine
39. trimeprazine
40. chlorcylizine
41.triphennamine
42. codeine
43. cyproheptadine
44. phenyltoloxamine
45. clemastine
46.famotidine
47. hydrocodone
48. methscopolamine
49. neostigmine
50. gabapentin
51. lithium compounds
52. dopamine
53. bromocriptine
54. carbamazepine
55. desipramine
56. nortriptyline
57. quinidine
58. procainamide
59. ranitidine
60. quinine

The excipients commonly used in the formulations are as follows: Microcrystalline cellulose, lactose, mannitol, sucrose, polyvinylpyrrolidone (PVP), HPMC, and cellulose compounds as diluents; magnesium aluminum silicate (MAS), and xanthan gum as anti-clumping agents; starch, hydroxypropylmethylcellulose (HPMC E-10) and xanthan gum as binders; sucrose, saccharin sodium, sucralose, and monoammoniumglycrhizinate as sweetening agents; calcium phosphate as hardness enhancer; talc and colloidal silicon dioxide as a glidants; magnesium stearate as a lubricant and citric acid and grape flavor as flavoring agents. Other excipients may be used without departing from the spirit and scope of the present invention. Active ingredients not present as tannate salt complexes also can be included in the formulation.

The pharmaceutically acceptable liquid used to wet the dry powder mixture of active ingredient salt or free base and tannic acid is preferably water. However, other pharmaceutically acceptable liquids may be substituted for water such as isopropyl alcohol, ethanol, glycerin, propylene glycol, mineral oil or mixtures thereof. The addition of the pharmaceutically acceptable liquid leads to the dissociation of the active ingredient salt into its free-base and conjugate acid forms which facilitates the formation of the tannate salt complex of the active ingredient.

The following examples illustrate the conversion process and subsequent incorporation of the tannate salt complexes into suitable solid dosage forms. The following examples are only intended to illustrate the present invention, and are not intended to limit its scope. Many other active pharmaceutical ingredients may be converted to the tannate salt form in like manner.

### EXAMPLE 1

Preparation of a Phenvlephrine Tannate, Chlorpheniramine Tannate, and Hyoscyamine Tannate Combination Product:

| Ingredient | % In Total, w/v | Amount (g) |
|---|---|---|
| Chlorpheniramine Maleate | 2.00% | 176.00 |
| Phenylephrine HCl | 5.00% | 440.00 |
| Hyoscyamine Sulfate | 0.06% | 5.50 |
| Tannic Acid, USP | 10.19% | 896.91 |
| Magnesium Aluminum | 2.00% | 176.00 |
| Silicate (MAS), NF | | |
| Methocel E-10M (HPMC) | 50.00% | 4040.00 |
| Lactose | 28.70% | 2525.19 |
| FD&C Blue #1 Aluminum | 0.30% | 24.00 |
| Lake | | |
| Colloidal Silicon Dioxide | 0.50% | 40.00 |
| Magnesium Stearate, NF | 1.25% | 100.00 |
| Purified Water, USP˜* | NA | 150.00 |
| Total | 100.00% | 8000.0 |

An excess of 10% is added to raw materials used in the wet granulation to correct for losses.

Tablets utilizing the above formulation were prepared as follows. The tannic acid, MAS, chlorpheniramine maleate, phenylephrine HCl, and hyoscyamine sulfate dry powders were mixed for a period of 10 minutes in a planetary mixer to obtain a uniform blend. While continuing to mix, 150 mL of purified water was sprayed onto the dry powder blend. The conversion process occurred as soon as the tannic acid and API salts were moistened. Mixing was continued for an additional 20 minutes. At this point, the conversion product had a dough-like consistency, but was still able to be poured from the bowl.

To a separate mixing vessel, most preferably a paddle blender, the following dry powders were added: methocel E-10M (HPMC) and lactose. The conversion product was poured evenly over these dry powders, and the mixture was then blended for 20 minutes. At this point, a product resembling a typical wet granulation was obtained. The mixture was then dried, milled, and the remainder of the excipients were added. The final blend was then processed into 400 mg tablets using techniques well known in the art.

### EXAMPLE 2

Preparation of a Phenylephrine Tannate, Hvoscyamine Tannate, and Guaifenesin Combination Product:

| Ingredient | % In Total, w/v | Amount (g) |
|---|---|---|
| Guaifenesin | 25.00% | 2000.00 |
| Phenylephrine HCl | 6.25% | 550.00 |
| Hyoscyamine Sulfate | 0.06% | 5.50 |
| Tannic Acid, USP | 6.31% | 555.50 |
| Magnesium Aluminum | 2.00% | 176.00 |
| Silicate (MAS), NF | | |
| Methocel E-10M (HPMC) | 35.88% | 3157.42 |
| Lactose | 22.44% | 1975.19 |
| FD&C Blue #1 Aluminum | 0.30% | 24.00 |
| Lake | | |
| Colloidal Silicon Dioxide | 0.50% | 40.00 |
| Magnesium Stearate, NF | 1.25% | 100.00 |
| Purified Water, USP˜* | NA | 150.00 |
| Total | 100.00% | 8000.0 |

An excess of 10% is added to raw materials used in the wet granulation to correct for losses.

Tablets utilizing the above formulation are prepared as follows. The tannic acid, MAS, phenylephrine HCl, and hyoscyamine sulfate dry powders are mixed for a period of 10 minutes in a planetary mixer to obtain a uniform blend. While continuing to mix, 150 mL of purified water is sprayed onto the dry powder blend. The conversion process occurs as soon as the tannic acid and API salts are moistened. Mixing is continued for an additional 20 minutes. At this point, the conversion product has a dough-like consistency, but is still able to be poured from the bowl.

To a separate mixing vessel, most preferably a paddle blender, the following dry powders are added: methocel E-10M (HPMC) and lactose. The conversion product is poured evenly over these dry powders, and the mixture is then blended for 20 minutes. At this point, a product resembling a typical wet granulation is obtained. The mixture is then dried and milled as needed. The remainder of the excipients and the guaifenesin are added and the mixture is blended for an additional 20 minutes. The final blend is then processed into tablets, capsules, or other solid dosage forms as required using techniques well known in the art

The ingredients of typical tablet formulations containing tannate salts of active ingredients prepared using the method of the present invention is detailed above. Tablets may be prepared from this formulation using well known conventional manufacturing techniques. Many equivalents to the materials contained in the formulation above may be substituted without departing from the spirit and scope of the present invention.

1. A process for the conversion of at least one active pharmaceutical ingredient into its tannate salt complex for incorporation into a therapeutic tablet, capsule or other solid dosage form, the process comprising the steps of: (a) mixing the salt or free base of the active pharmaceutical ingredient, tannic acid, and an anti-clumping agent to form a powder mixture; (b) adding a pharmaceutically acceptable liquid to the powder mixture of step (a) to form a moistened blend; (c) separately mixing additional pharmaceutically acceptable excipients to generate a second powder blend; (d) combining the powder blend of step (c) with the moistened blend of step (b) to form a granulate; (e) processing the granulate of step (d) into tablet, capsule, or other solid dosage forms using techniques well known in the art

2. A process according to claim 1, wherein step (e) includes the additional requirement of adding additional pharmaceutically acceptable excipients.

3. The process according to claim 1, wherein the active pharmaceutical ingredient is selected from the group consisting of: (1) carbinoxamine (2) chlorpheniramine (3) pyrilamine (4) pheniramine (5) phenindamine (6) diphenhydramine (7) bromodiphenhydramine (8) triplennamine (9) brompheniramine (10) loratadine (11) desloratidine (12) fexofenadine (13) carbetapentane (14) dextromethorphan (15) phenylephrine (16) pseudoephedrine (17) ephedrine (18) oxycodone (19) morphine (20) physostigmine (21) cimetidine (22) amantidine (23) fluvoxamine (24) sertraline (25) chlorpromazine (26) imipramine (27) amitryptyline (28) prochlorperazine (29) cetirizine (30) hydroxyzine (31) promethazine (32) acrivastine (33) triprolidine (34) meclizine (35) dimenhydrinate (36) dexchlorpheniramine (37) doxylamine (38) diphenylpyrilamine (39) trimeprazine (40) chlorcylizine (41) triphennamine (42) codeine (43) cyproheptadine (44) phenyltoloxamine (45) clemastine (46) famotidine (47) hydrocodone (48) methscopolamine (49) ncostigmine (50) gabapentin (51) lithium compounds (52) dopamine (53) bromocriptine (54) carbamazepine (55) desipramine (56) nortriptyline (57) quinidine (58) procainamide (59) ranitidine (60) quinine

4. The process according to claim 1 wherein the active pharmaceutical ingredients are provided as the bitartrate, maleate, citrate, chloride, bromide, acetate or sulfate salt.

5. The process according to claim 1 wherein the anti-clumping agent added to step (a), is selected from the group consisting of magnesium aluminum silicate, xanthan gum and cellulose compounds.

6. The process according to claim 1 wherein the anti-clumping agent added to step (a) is magnesium aluminum silicate.

7. The process according to claim 1 wherein the pharmaceutically acceptable liquid in step (b) is selected from the group consisting of purified water, isopropyl alcohol, ethanol, glycerin, propylene glycol, mineral oil and mixtures thereof.

8. The process according to claim 7 wherein the pharmaceutically acceptable liquid is purified water.

9. The process according to claim 1 wherein the tannic acid is derived from a natural source.

10. The process according to claim 1 wherein the tannic acid is derived from a synthetic process.

11. The process according to claim 1 wherein step (e) includes the additional requirement of adding a non-tannate active pharmaceutical ingredient.

12. The process according to claim 1 wherein step (d) includes the step of pouring the moistened blend of step (b) over the powder blend of step (c).
